(19) United States
(12) Patent Application Publication
(10) Pub. No.: US 2018/0242928 A1
(43) Pub. Date: Aug. 30, 2018

(54) SYSTEM AND APPARATUS FOR SURGERY PROGRESS MANAGEMENT

(71) Applicant: MEDICAROID CORPORATION, Kobe-shi (JP)

(72) Inventors: Yasuhiro Kochi, Cupertino, CA (US); Leila Bahreinian, Los Gatos, CA (US); Terry Chang, San Jose, CA (US); Yasuyuki Fukuda, Nishinomiya (JP)

(73) Assignee: MEDICAROID CORPORATION, Kobe-shi (JP)

(21) Appl. No.: 15/905,095

(22) Filed: Feb. 26, 2018

Related U.S. Application Data
(60) Provisional application No. 62/464,730, filed on Feb. 28, 2017.

(51) Int. Cl. A61B 6/04; G16H 30/20; G06K 7/14; G06K 7/10; G06K 19/06; G06K 19/077; A61G 13/04; A61G 13/06

(52) U.S. Cl. CPC A61B 6/0442; G16H 30/20; G06K 7/1413; G06K 7/10366; H04L 67/12; G06K 19/07758; A61G 13/04; A61G 13/06; G06K 19/06028

(57) ABSTRACT

A surgery-progress management system according to one or more embodiments may include: a robotic operating table including a table top on which to place a patient and a robotic arm which comprises a plurality of joints and supports the table top; and a surgery-progress management apparatus that manages progress of surgery. The surgery-progress management apparatus may include a communication unit that communicates information with the robotic operating table, and an information processing unit that acquires information of the robotic operating table from the robotic operating table through the communication unit and generates surgery progress information based on the acquired information of the robotic operating table.

### CROSS REFERENCE TO RELATED APPLICATIONS

This application claims the benefit of U.S. Provisional Application No. 62/464,730 filed on Feb. 28, 2017, entitled “SYSTEM AND APPARATUS FOR SURGERY PROCESS MANAGEMENT”, the entire contents of which are incorporated herein by reference.

### BACKGROUND

The disclosure relates to a surgery-progress management system and a surgery-progress management apparatus.

Heretofore, surgery progress management apparatuses that manage the progress of surgery have been known (see e.g., Japanese Patent Application Publication No. 201 4-1 841 26 (Patent Literature 1)).

Patent Literature 1 discloses a surgery-progress notification apparatus that manages the progress of a surgery by using instruments used in the surgery. The surgery-progress notification apparatus disclosed in Patent Literature 1 detects the on and off states of the switches of the power sources for instruments provided in an operating room. The surgery-process notification apparatus is able to determine the progress of a surgery based on the detected information about the instruments. The instruments for which information is detected may include a clock, an echo machine, shadowless lamps, an electrocauterizer, a bipolar electrocoagulator, an electrocardiogram monitor, a coagulating incision device, an a-line monitor, a cleaning-suction device, a blood pressure monitor, a gauze counter, an infusion pump, a radiographic imaging apparatus, a warming device for liquid or blood to be transfused, a pneumatic compression device, an oxygen saturation monitor, a bladder temperature monitor, a mattress controller, a warming device, an anesthetizing device, a laid sheet-type mattress, and a drape-type body temperature keeper.

### SUMMARY

A surgery-progress management system according to one or more embodiments may include: a robotic operating table including a table top on which to place a patient and a robotic arm which comprises a plurality of joints and supports the table top; and a surgery-progress management apparatus that manages progress of surgery. In one or more embodiments, the surgery-progress management apparatus may include a communication unit that communicates information with the robotic operating table, and an information processing unit that acquires information of the robotic operating table from the robotic operating table through the communication unit and generates surgery progress information based on the acquired information of the robotic operating table.

A surgery-progress management apparatus according to one or more embodiments may include: a communication unit that communicates information with a robotic operating table including a table top on which to place a patient and a robotic arm which comprises a plurality of joints and supports the table top; and an information processing unit that acquires information of the robotic operating table from the robotic operating table through the communication unit and generates surgery progress information based on the acquired information on the robotic operating table.

### BRIEF DESCRIPTION OF DRAWINGS

FIG. 1 is a diagram illustrating surgery-progress management systems according to first and second embodiments;

FIG. 2 is a perspective view illustrating robotic operating tables according to first and second embodiments;

FIG. 3 is a block diagram illustrating the robotic operating tables according to first and second embodiments;

FIG. 4A to FIG. 4E are views illustrating preset positions of a table of the robotic operating tables according to first and second embodiments;

FIG. 5 is a diagram illustrating generation of progress information by surgery-progress management apparatuses according to first and second embodiments;

FIG. 6 is a flowchart illustrating a progress-information generation process by the surgery-progress management apparatus according to a first embodiment;

FIG. 7 is a diagram illustrating updating of a scheduled surgery end time by the surgery-progress management apparatus according to a second embodiment;

FIG. 8 is a flowchart illustrating a surgery-information generation process by the surgery-progress management apparatus according to a second embodiment;

FIG. 9 is a flowchart illustrating a scheduled-end-time update process by the surgery-progress management apparatus according to a second embodiment; and

FIG. 10 is a flowchart illustrating a work-cue informing process by the surgery-progress management apparatus according to a second embodiment.

### DETAILED DESCRIPTION

Embodiments are described with reference to drawings, in which the same constituents are designated by the same reference numerals and duplicate explanation concerning the same constituents may be omitted for brevity and ease of explanation. The drawings are illustrative and exemplary in nature and provided to facilitate understanding of the illustrated embodiments and may not be exhaustive or limiting. Dimensions or proportions in the drawings may not be to scale, and are not intended to impose restrictions on the disclosed embodiments. For this reason, specific dimensions and the like should be interpreted with the accompanying descriptions taken into consideration. In addition, the drawings may include parts whose dimensional relationship and ratios are different from one drawing to another.

Prepositions, such as “on”, “over” and “above” may be defined with respect to a surface, for example a layer surface, regardless of the orientation of the surface in space.

### First Embodiment

### Configuration of Surgery-Progress Management System

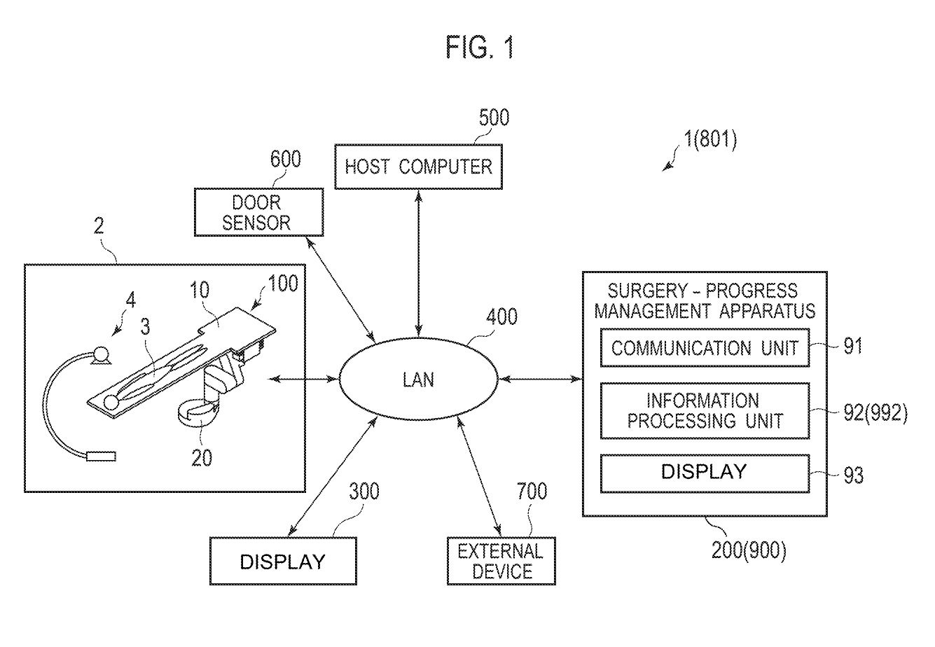

The configuration of a surgery-progress management system 1 according to an embodiment is explained with reference to FIG. 1 to FIG. 5.

As illustrated in FIG. 1, the surgery-progress management system 1 is a system that manages the progress of surgery performed on a patient 3 in an operating room 2. The surgery-progress management system 1 may be installed inside a hospital in which the operating room 2 is provided. As used herein, the term “surgery” may be defined as including not only the process of cutting the body of a patient and performing a therapeutic procedure but may also include a series of processes from the entry of the patient into the operating room to the exit of the patient from the operating room.

The surgery-progress management system 1 includes a robotic operating table 100, a surgery-progress management apparatus 200, and a display 300. The robotic operating table 100 may be installed inside the operating room 2. The surgery-progress management apparatus 200 and the display 300 may be installed outside the operating room 2. Also, the display 300 may be installed in a different room from a room in which the surgery-progress management apparatus 200 is installed. For example, the surgery-progress management apparatus 200 may be installed in a control room next to the operating room 2, while the display 300 may be installed in a nurse station.

In the surgery-progress management system 1, the robotic operating table 100, the surgery-progress management apparatus 200, and the display 300 are connected to an Local Area Network (LAN) 400 inside the hospital in which the operating room 2 is provided. The LAN 400 is connectable to an external network outside the hospital. A host computer 500, a door sensor 600, and an external device 700 are also connected to the LAN 400. The robotic operating table 100, the surgery-progress management apparatus 200, the display 300, the host computer 500, the door sensor 600, and the external device 700 are communicatively connected to each other through the LAN 400.

The host computer 500 may be a computer that manages a system in the hospital including managing surgery information, patient information, and other information. The door sensor 600 is a sensor that detects opening and closing of the door of the operating room 2 through which the patient 3 enters and exits the operating room 2. The external device 700 may be a mobile terminal held or otherwise operated by a worker that performs work in the operating room 2 after surgery (e.g. cleaner), and has at least an information displaying function. Although one display 300 and one external device 700 are illustrated in FIG. 1, multiple displays 300 and multiple external devices 700 may be provided in accordance with one or more embodiments.

### Configuration of Robotic Operating Table

In one or more embodiments, the robotic operating table 100 may be used as an operating bed on which to perform operations in a setting such as a surgery setting or internal medicine setting. The robotic operating table 100 may be provided in the operating room 2. A radiographic imaging apparatus 4 that captures a radiographic projection image of the patient 3 may be provided in the operating room 2. The operating room 2 may be a hybrid operating room.

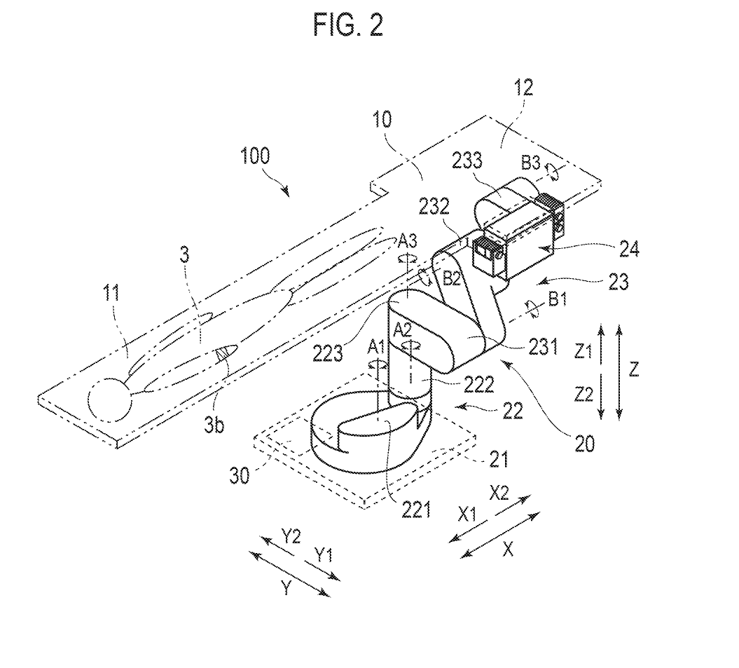

As illustrated in at least FIG. 2, the robotic operating table 100 may include a table 10 on which to place the patient 3, a robotic arm 20, and a control unit 30. The robotic arm is an articulated robotic arm, which includes a plurality of joints.

The table 10 may be formed in the shape of a substantially rectangular flat plate. Also, the upper surface of the table 10 may be formed to be substantially flat. While the table 10 is rotatable about an axis extending in the vertical direction (Z direction), the horizontal direction along the longitudinal direction of the table 10 is defined as the X direction and the horizontal direction along the transverse direction of the table 10 is defined as the Y direction in FIG. 2. In other words, the X direction and the Y direction represent directions with reference to the table 10.

The table 10 may include a radiolucent part 11 that is transparent to X-rays, and a support part 12 supporting the radiolucent part 11.

The patient 3 is placed on the radiolucent part 11 of the table 10. The radiolucent part 11 is disposed on the X1 direction side of the table 10. The radiolucent part 11 is formed in a substantially rectangular shape. The radiolucent part 11 is made of a radiolucent material. The radiolucent part 11 is made of a carbon material (graphite), for example. The radiolucent part 11 is made of a carbon fiber reinforced plastic (CFRP), for example. In this way, an image of the patient 3 can be captured using X-rays while the patient 3 is placed on the radiolucent part 11.

The support part 12 of the table 10 is connected to the robotic arm 20. The support part 12 is disposed on the X2 direction side of the table 10. The support part 12 is formed in a substantially rectangular shape. The support part 12 supports the radiolucent part 11. The support part 12 is made of a material smaller in radiolucency than the material the radiolucent part 11 is made of. The support part 12 may be made of metal. For example, the support part 12 may be made of a steel material or an aluminum material.

The table 10 is moved by the robotic arm 20. Specifically, the table 10 is movable in the X direction, which is a horizontal direction, in the Y direction, which is the horizontal direction perpendicular to the X direction, and in the Z direction, which is perpendicular to the X direction and the Y direction and is the vertical direction. Moreover, the table 10 is rotatable (capable of being caused to roll) about an axis extending in the X direction. The table 10 is also rotatable (capable of being caused to pitch) about an axis extending in the Y direction. The table 10 is also rotatable (capable of being caused to yaw) about an axis extending in the Z direction.

The robotic arm 20 moves the table 10. One end of the robotic arm 20 is supported on a base 21 fixed to the floor, while the opposite end supports the table 10. Specifically, the one end of the robotic arm 20 is supported on the base 21 to be rotatable about a base rotation axis (rotation axis A1) extending in the vertical direction (Z direction). The base 21 is a base buried in and fixed to the floor. The base 21 is provided substantially at the center of the range of movement of the table 10 in a plan view (as seen from the Z direction). Also, the opposite end of the robotic arm 20 supports the table 10 at a position near its one end in the longitudinal direction of the table 10 (X direction). Specifically, the opposite end of the robotic arm 20 supports the support part 12, which is disposed on the one end side of the table 10 in the longitudinal direction of the table 10.

The robotic arm 20 includes a horizontal articulated assembly 22, a vertical articulated assembly 23, and a pitch mechanism 24. The horizontal articulated assembly 22 includes horizontal joints 221, 222, and 223. The vertical articulated assembly 23 includes vertical joints 231, 232, and 233. The horizontal joints 221 to 223 and the vertical joints 231 to 233 may be examples of “joints” in one or more recited embodiments.

The robotic arm 20 moves the table 10 with seven degrees of freedom. Specifically, with the horizontal articulated assembly 22, the robotic arm 20 has three degrees of freedom to rotate about the rotation axis A1, extending in the vertical direction (Z direction), rotate about a rotation axis A2 extending in the vertical direction, and rotate about a rotation axis A3 extending in the vertical direction. Further, with the vertical articulated assembly 23, the robotic arm 20 has three degrees of freedom to rotate about a rotation axis B1 extending a horizontal direction, rotate about a rotation axis B2 extending in the horizontal direction, and rotate about a rotation axis B3 extending in the horizontal direction. Furthermore, with the pitch mechanism 24, the robotic arm 20 has one degree of freedom to allow the table 10 to pitch about a rotation axis extending in the transverse direction of the table 10 (Y direction).

One end of the horizontal articulated assembly 22 is supported on the base 21. Moreover, the opposite end of the horizontal articulated assembly 22 supports one end of the vertical articulated assembly 23. The horizontal joint 221 of the horizontal articulated assembly 22 rotates the table 10 about the rotation axis A1, extending in the vertical direction (Z direction). The horizontal joint 222 of the horizontal articulated assembly 22 rotates the table 10 about the rotation axis A2, extending along the vertical direction. The horizontal joint 223 of the horizontal articulated assembly 22 rotates the table 10 about the rotation axis A3, extending in the vertical direction.

The one end of the vertical articulated assembly 23 is supported on the horizontal articulated assembly 22. Moreover, the opposite end of the vertical articulated assembly 23 supports the pitch mechanism 24. The vertical joint 231 of the vertical articulated assembly 23 rotates the table 10 about the rotation axis B1, extending in the X direction. The vertical joint 232 of the vertical articulated assembly 23 rotates the table 10 about the rotation axis B2, extending in the X direction. The vertical joint 233 of the vertical articulated assembly 23 rotates the table 10 about the rotation axis B3, extending in the X direction.

The gap between each pair of adjacent joints is shorter than the length of the table 10 in the transverse direction of the table 10 (Y direction). Specifically, the gap between the rotation axis Al and the rotation axis A2, the gap between the rotation axis A2 and the rotation axis A3, the gap between the rotation axis A3 and the rotation axis B1, the gap between the rotation axis B1 and the rotation axis B2, and the gap between the rotation axis B2 and the rotation axis B3 are each shorter than the length of the table 10 in the transverse direction of the table 10.

One end of the pitch mechanism 24 is supported on the vertical articulated assembly 23. Moreover, the opposite end of the pitch mechanism 24 supports the support part 12 of the table 10. Also, the pitch mechanism 24 is disposed near one side of the table 10 in the transverse direction of the table 10 (Y direction). Specifically, the pitch mechanism 24 is disposed near the end of the table 10 in the Y1 direction.

The control unit 30 may comprise control circuitry including, for example, a Central Processing Unit (CPU) 30a and a storage device 30b, such as a hard disk drive or a flash memory. The control unit 30 may be disposed inside the base 21 and controls movement of the table 10 by the robotic arm 20. Specifically, the control unit 30 moves the table 10 by controlling drive of the robotic arm 20 based on an operation by a medical person (operator). The storage device 30b stores an application program for transmitting operation commands to the robotic arm 20 to move the table 10 based on information inputted by a medical person by using an operation unit 80 to be explained later. In other words, the application program may comprise software for causing the robotic arm 20 to operate according to a received operation command. The storage device 30b also stores preset-position information and other information that is registered using the operation unit 80, as explained later.

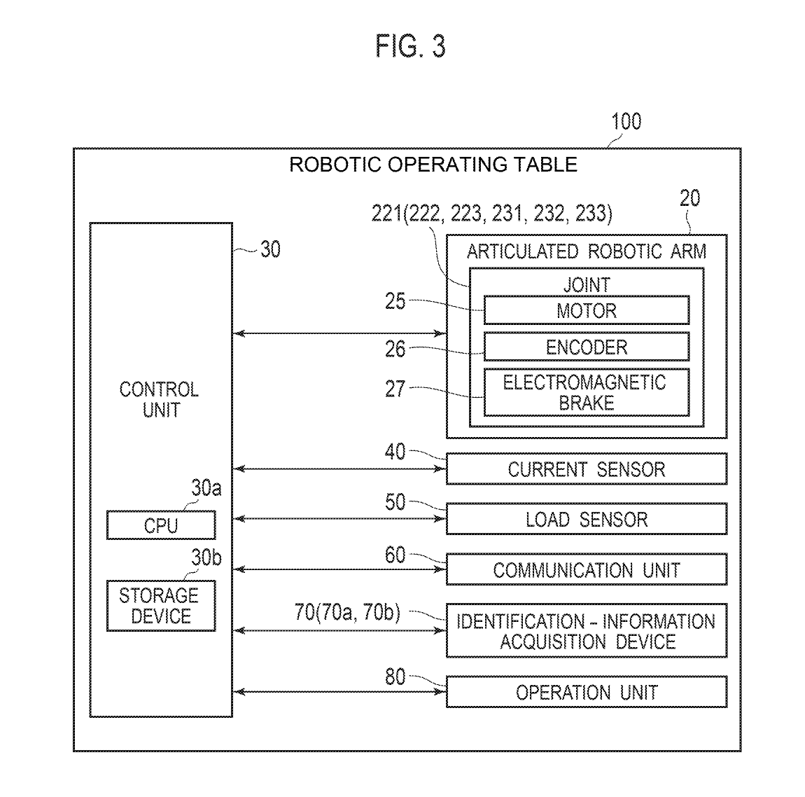

As illustrated in at least FIG. 3, the joints of the robotic arm 20 (horizontal joints 221 to 223 and vertical joints 231 to 233) each include a motor 25, an encoder 26, and an electromagnetic brake 27. The motor 25 is a drive source for the joint to rotate the table 10. The motor 25 includes a servomotor. The encoder 26 measures the amount of rotation of the motor 25. Meanwhile, the robotic operating table 100 generates position information on the table 10 based on the result of the measurement by the encoder 26. The electromagnetic brake 27 is a negative actuated-type electromagnetic brake that stops the rotation by the motor 25. Specifically, the electromagnetic brake 27 cancels braking of the motor 25 when the motor 25 is energized, and brakes the motor 25 when the motor 25 is de-energized.

The robotic operating table 100 also includes current sensors 40, a load sensor 50, a communication unit 60, an identification-information acquisition device 70, and the operation unit 80.

The current sensors 40 are sensors that may be provided individually for the joints of the robotic arm 20 and that acquire the current values of the motors 25 in the respective joints. The robotic operating table 100 acquires the current values of the motors 25 in the joints of the robotic arm 20 from the current sensors 40. Moreover, the robotic operating table 100 generates load information on a load received by the robotic arm 20 from the table 10 based on the result of the acquisition of the current values of the motors 25.

The load sensor 50 is a sensor that detects a load received by the table 10. Specifically, the load sensor 50 is a sensor that detects whether or not the patient 3 is placed on the table 10.

The communication unit 60 communicates information with the surgery-progress management apparatus 200. The robotic operating table 100 transmits the position information on the table 10, the load information, the result of the detection by the load sensor 50, and so on to the surgery-progress management apparatus 200 through the communication unit 60. The robotic operating table 100 is connected to the LAN 400 through the communication unit 60.

The identification-information acquisition device 70 is a device that acquires identification information attached to the patient 3. The identification-information acquisition device 70 may be mounted, such as to the table 10. The identification-information acquisition device 70 is capable of acquiring the identification information from a patient identification band 3a (see FIG. 2) attached to the patient 3 from a position around the table 10. The identification information may be embodied in a barcode attached to the patient identification band 3a or may be stored in an RFID tag attached to the patient identification band 3a. The identification-information acquisition device 70 may be a barcode reader 70a in the case where the identification information is a barcode. The identification-information acquisition device 70 may be an RFID reader 70b in the case where the identification information is stored in an RFID tag.

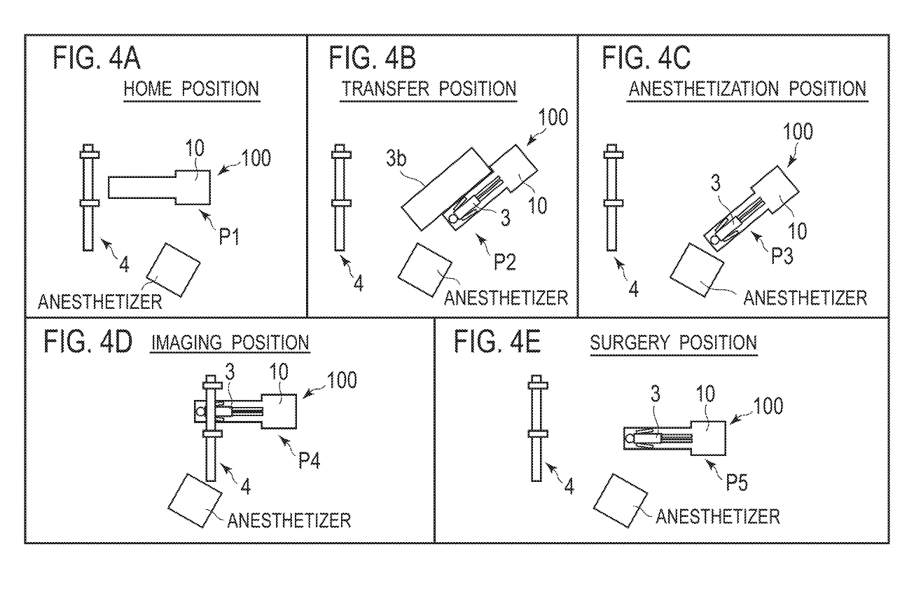

The operation unit 80 is a device that receives operations for moving the table 10 by a medical person (operator). As illustrated in FIG. 4A to FIG. 4E, the control unit 30 moves the table 10 to a position such as a home position P1, a transfer position P2, an anesthetization position P3, an imaging position P4, or a surgery position P5 by using the robotic arm 20 based on the result of an operation on the operation unit 80 by the medical person. Note that the home position P1 is a default position of the table 10. The transfer position P2 is a position for transferring the patient 3 between a stretcher 3b and the table 10. Specifically, the transfer position P2 is a position for transferring the patient 3 from the stretcher 3b to the table 10 and a position for transferring the patient 3 from the table 10 to the stretcher 3b. Also, the anesthetization position P3 is a position for anesthetizing the patient 3. Also, the imaging position P4 is a position for capturing an image with the radiographic imaging apparatus 4. Also, the surgery position P5 is a position for performing a surgery.

The robotic operating table 100 is capable of registering positions such as the home position P1, the transfer position P2, the anesthetization position P3, the imaging position P4, and the surgery position P5 as preset positions. Specifically, the robotic operating table 100 registers a predetermined position as a preset position in response to a register command issued from the operation unit 80 with the table 10 placed at the predetermined position in advance. In doing so, the robotic operating table 100 causes the storage device 30b of the control unit 30 to store the position information on the table 10 and posture information on the robotic arm 20 as the preset position. The robotic operating table 100 in a first embodiment registers the home position P1, the transfer position P2, the anesthetization position P3, the imaging position P4, and the surgery position P5 as preset positions. The robotic operating table 100 may energize the motors 25, thereby cancelling the braking of the motor 25 by the electromagnetic brake 27 and allowing the robotic arm 20 to move the table 10, only while a medical person is performing a moving operation by using the operation unit 80. Moreover, the robotic operating table 100 de-energizes the motor 25, thereby braking the motor 25 with the electromagnetic brake 27 and stopping the robotic arm 20, when the table 10 is placed at a preset position.

### Configuration of Surgery-Progress Management Apparatus

As illustrated in at least FIG. 1, the surgery-progress management apparatus 200 is an apparatus that manages the progress of a surgery. The surgery-progress management apparatus 200 includes a communication unit 91, an information processing unit 92, and a display 93.

The communication unit 91 communicates information with the robotic operating table 100. The surgery-progress management apparatus 200 is connected to the LAN 400 through the communication unit 91.

In a first embodiment, the information processing unit 92 acquires the position information on the table 10 from the robotic operating table 100 through the communication unit 91 and generates surgery progress information based on the acquired position information on the table 10. The position information on the table 10 includes home-position information indicating that the table 10 has been placed at the home position P1, transfer-position information indicating that the table 10 has been placed at the transfer position P2, anesthetization-position information indicating that the table 10 has been placed at the anesthetization position P3, imaging-position information indicating that the table 10 has been placed at the imaging position P4, and surgery-position information indicating that the table 10 has been placed at the surgery position P5.

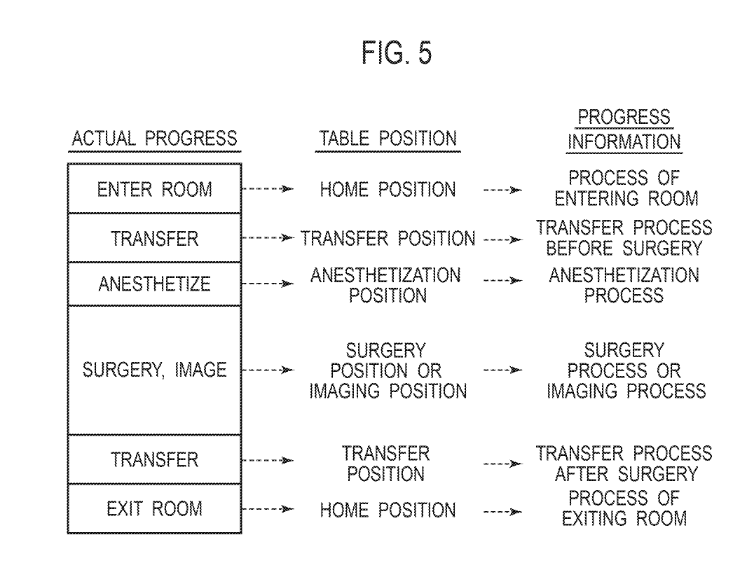

As illustrated in at least FIG. 5, the information processing unit 92 may generate information indicating that the surgery has progressed to a process of entering the room or information indicating that the surgery has progressed to a process of exiting the room, as surgery progress information based on the home-position information. The information processing unit 92 may further generate, before the surgery begins, information indicating that the surgery has progressed to a transfer process, as surgery progress information based on the transfer-position information. Alternatively, or additionally, the information processing unit 92 may further generate, after the surgery, information indicating that the surgery has progressed to a transfer process, as surgery progress information based on the transfer-position information. In other words, the information processing unit 92 generates information indicating that the surgery is about to start soon or information indicating that the surgery is about to end soon, as surgery progress information based on the transfer-position information.

The information processing unit 92 may further generate information indicating that the surgery has progressed to an anesthetizing process, as surgery progress information based on the anesthetization-position information. The information processing unit 92 may further generates information indicating that the surgery has progressed to an imaging process, as surgery progress information based on the imaging-position information. Also, the information processing unit 92 may generate information indicating that the surgery has progressed to a surgery process, as surgery progress information based on the surgery-position information.

Further, in a first embodiment, the information processing unit 92 may acquire the load information from the robotic operating table 100 through the communication unit 91, and may generate surgery progress information based on the acquired load information in addition to the position information on the table 10. The information processing unit 92 may generate information indicating that the transfer of the patient 3 before the surgery has been completed, as surgery progress information based on the load information indicating that the patient 3 has been placed on the table 10. The information processing unit 92 may further generate information indicating that the transfer of the patient 3 after the surgery has been completed, as surgery progress information based on the load information indicating that the patient 3 is not placed on the table 10.

Further, the information processing unit 92 acquires the result of the detection by the load sensor 50 from the robotic operating table 100 through the communication unit 91, and may generate surgery progress information based on the acquired result of the detection by the load sensor 50 in addition to the position information on the table 10. The information processing unit 92 may generate information indicating that the transfer of the patient 3 before the surgery has been completed, as surgery progress information based on the result of the detection by the load sensor 50 indicating that the patient 3 has been placed on the table 10. Also, the information processing unit 92 may generate information indicating that the transfer of the patient 3 after the surgery has been completed, as surgery progress information based on the result of the detection by the load sensor 50 indicating that the patient 3 is not placed on the table 10.

The information processing unit 92 may further acquire the result of the detection by the door sensor 600 from the robotic operating table 100 through the communication unit 91, and may generate surgery progress information based on the acquired result of the detection by the door sensor 600 in addition to the position information on the table 10. The information processing unit 92 may generate information indicating that the entry of the patient 3 into the operating room 2 has been completed, as surgery progress information based on the result of the detection by the door sensor 600 indicating that the door has been opened before the surgery. Also, the information processing unit 92 may generate information indicating that the exit of the patient 3 from the operating room 2 has been completed, as surgery progress information based on the result of the detection by the door sensor 600 indicating that the door has been opened after the surgery.

In a first embodiment, the information processing unit 92 performs control for displaying information that is based on the surgery progress information generated on the display 93. Accordingly, a medical person (e.g. operator) or other person, can determine the progress of the surgery in the room in which the surgery-progress management apparatus 200 is installed. The information processing unit 92 may also transmit the generated surgery progress information to the display 300 and the external device 700 through the communication unit 91. The display 300 and the external device 700 display information that is based on the received surgery progress information. In this way, a medical person (e.g. operator) or other person, can determine the progress of the surgery in the room in which the display 300 is installed, and the worker having the external device 700 can determine the progress of the surgery with the external device 700. Meanwhile, the surgery-progress management apparatus 200 provides the generated surgery progress information through the communication unit 91 to the external device 700 in response to a request therefrom.

In a first embodiment, the surgery-progress management apparatus 200 also functions as a patient identification apparatus that identifies the patient 3. The surgery-progress management apparatus 200 acquires patient information from the host computer 500 through the communication unit 91 and acquires the identification information on the patient 3 from the robotic operating table 100.

The information processing unit 92 of the surgery-progress management apparatus 200 compares the identification information acquired from the robotic operating table 100 and the patient information acquired from the host computer 500. The information processing unit 92 may further generate warning information if the identification information and the patient information do not match each other. The information processing unit 92 may further transmit the generated warning information to the robotic operating table 100. The robotic operating table 100 may in turn be informed of a warning with light, a sound, a message, etc. based on the warning information.

### Progress-Information Generation Process

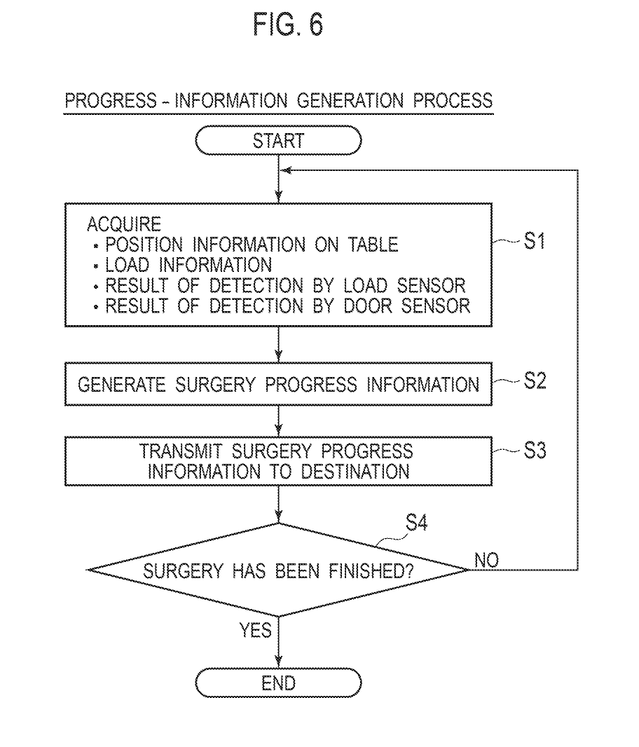

Next, a progress-information generation process by the surgery-progress management apparatus 200 in a first embodiment is explained with reference to a flowchart in FIG. 6. The information processing unit 92 may perform each process step in the flowchart.

As illustrated in at least FIG. 6, in step S1, the position information on the table 10, the load information, the result of the detection by the load sensor 50, and the result of the detection by the door sensor 600 may be acquired through the communication unit 91.

In step S2, surgery progress information may be generated based only on the position information on the table 10 or based on at least one of the position information on the table 10, the load information, the result of the detection by the load sensor 50, and the result of the detection by the door sensor 600.

In step S3, the generated surgery progress information is transmitted to predetermined destinations (e.g. display 93, display 300, and external device 700). As a result, information that is based on the surgery progress information is displayed at the destinations.

In step S4, it may be determined whether or not the surgery has been finished. If it is determined that the surgery has not been finished (S4=“NO”), the process returns to step S1, and the processes of steps S1 to S3 are repeated. On the other hand, if it is determined that the surgery has been finished (S4=“YES”), the progress-information generation process is terminated.

### Advantageous Effects of First Embodiment

In accordance with a first embodiment, the following advantageous effects may be offered.

In a first embodiment, as explained above, the surgery-progress management apparatus 200 may includes the communication unit 91, which communicates information with the robotic operating table 100, and the information processing unit 92, which acquires the position information on the table 10 from the robotic operating table 100 through the communication unit 91 and generates surgery progress information based on the acquired position information on the table 10. In this way, the surgery progress information can be generated based on the position information on the table 10 of the robotic operating table 100, which is used throughout the surgery while its state is changed with the progress of the surgery. Hence, the progress of the surgery can be accurately determined in real time based on the generated surgery progress information. Moreover, since the progress of the surgery can be accurately determined in real time, a worker, such as a cleaner, who cleans the operating room 2, can determine an appropriate time to stand by for the cleaning.

Also, in a first embodiment, as explained above, the position information on the table 10 includes the transfer-position information, indicating that the table 10 has been placed at the transfer position P2 for transferring the patient 3 between the stretcher 3b and the table 10. In this way, it is possible to determine that the patient 3 is about to be transferred from the stretcher 3b to the table 10, and therefore to accurately determine in real time that the surgery has progressed to the initial phase. It is also possible to determine that the patient 3 is about to be transferred from the table 10 to the stretcher 3b, and therefore accurately determine in real time that the surgery has progressed to the final phase.

Also, in a first embodiment, as explained above, the position information on the table 10 includes the anesthetization-position information, indicating that the table 10 has been placed at the anesthetization position P3 for anesthetizing the patient 3, the imaging-position information, indicating that the table 10 has been placed at the imaging position P4 for capturing an image with the radiographic imaging apparatus 4, and the surgery-position information, indicating that the table 10 has been placed at the surgery position P5 for performing surgery. In this way, it is possible to determine that that the patient 3 is about to be anesthetized or is being anesthetized when the position information on the table 10 is the anesthetization-position information. Also, it is possible to determine that an image is about to be captured or being captured when the position information on the table 10 is the imaging-position information. Also, it is possible to determine that the surgery is about to be performed or is being performed when the position information on the table 10 is the surgery-position information.

Also, in a first embodiment, as explained above, the information processing unit 92 generates information indicating that the surgery is about to end soon, as surgery progress information based on the transfer-position information. In this way, a worker, such as a cleaner, who cleans the operating room 2, can accurately determine an appropriate time to stand by for the cleaning based on the information indicating that the surgery is about to end soon.

Also, in a first embodiment, as explained above, the robotic operating table 100 is capable of registering the transfer position P2 as a preset position. In this way, the table 10 of the robotic operating table 100 can be placed at the transfer position P2 with a simple operation.

Also, in a first embodiment, as explained above, the robotic operating table 100 is capable of registering the anesthetization position P3, the imaging position P4, and the surgery position P5 as preset positions. In this way, the table 10 of the robotic operating table 100 can be placed at the anesthetization position P3, the imaging position P4, and the surgery position P5 with simple operations. Meanwhile, being capable of placing the table 10 at the imaging position P4 and the surgery position P5 with simple operations is useful especially in a case where the table 10 needs to be moved many times between the imaging position P4 and the surgery position P5 during the surgery.

Also, in a first embodiment, as explained above, the robotic operating table 100 registers a predetermined position as a preset position in response to a register command issued with the table 10 placed at the predetermined position in advance. In this way, a predetermined position desired by the user can be registered as a preset position with a simple and intuitive operation.

Also, in a first embodiment, as explained above, the robotic arm 20 includes joints (horizontal joints 221 to 223 and vertical joints 231 to 233). Further, each of the joints of the robotic arm 20 includes the motor 25 and the encoder 26, which measures the amount of rotation of the motor 25. Furthermore, the robotic operating table 100 generates the position information on the table 10 based on the result of the measurement by the encoder 26. In this way, the position information on the table 10 can be generated using the encoder 26, which measures the amount of rotation of the motor 25 and, which can prevent complication of the configuration for generating the position information on the table 10.

Also, in a first embodiment, as explained above, the robotic operating table 100 acquires the current value of the motor 25 in each of the joints of the robotic arm 20 (horizontal joints 221 to 223 and vertical joints 231 to 233) and generates the load information on the load received by the robotic arm 20 from the table 10 based on the result of the acquisition of the current value of the motor 25. Moreover, the information processing unit 92 acquires the load information from the robotic operating table 100 through the communication unit 91, and generates surgery progress information based on the acquired load information in addition to the position information on the table 10. In this way, whether or not the patient 3 is placed on the table 10 can be determined based on the load information. Thus, the progress of the surgery can be more accurately determined by generating surgery progress information based on not only the position information on the table 10 but also the load information.

Also, in a first embodiment, as explained above, the robotic operating table 100 includes the load sensor 50, which detects the load received by the table 10. Moreover, the information processing unit 92 acquires the result of the detection by the load sensor 50 from the robotic operating table 100 through the communication unit 91, and generates surgery progress information based on the acquired result of the detection by the load sensor 50 in addition to the position information on the table 10. In this way, it can be determined whether or not the patient 3 is placed on the table 10 based on the result of the detection by the load sensor 50. Thus, the progress of the surgery can be more accurately determined by generating surgery progress information based on not only the position information on the table 10 but also the result of the detection by the load sensor 50.

Also, in a first embodiment, as explained above, the information processing unit 92 acquires the result of the detection by the door sensor 600, which detects opening and closing of the door of the operating room 2 through the communication unit 91, and generates surgery progress information based on the result of the detection by the door sensor 600 in addition to the position information on the table 10. In this way, it is possible accurately determine that the patient 3 has entered the operating room 2 and that the patient 3 has exited the operating room 2, based on the position information on the table 10 and the result of the detection by the door sensor 600. Thus, the progress of the surgery can be more accurately determined.

Also, in a first embodiment, as explained above, the surgery-progress management system 1 includes the display 300, 93, which displays information that is based on the surgery progress information. Thus, the progress of the surgery can be easily determined based on the information displayed on the display 300, 93.

Also, in a first embodiment, as explained above, the surgery-progress management apparatus 200 provides the surgery progress information through the communication unit 91 to the external device 700 in response to a request therefrom. In this way, the progress of the surgery can be determined also with the external device 700, which is external to the surgery-progress management system. Determining the progress with the external device 700 is especially advantageous in a case where a worker who needs to determine the progress of the surgery (e.g. cleaner) can determine the progress of the surgery by using a terminal held or otherwise operated by the worker while at a location away from the surgery room.

Also, in a first embodiment, as explained above, the surgery-progress management apparatus 200 is communicatively connected to the host computer 500 through the communication unit 91. Moreover, the surgery-progress management apparatus 200 is capable of acquiring patient information from the host computer 500. In this way, patient information does not need to be manually input into the surgery-progress management apparatus 200. Acquiring patient information from the host computer 500 can reduce the work burden on the user.

Also, in a first embodiment, as explained above, the robotic operating table 100 includes the identification-information acquisition device 70, which acquires the identification information attached to the patient 3. Moreover, the information processing unit 92 compares the identification information acquired from the robotic operating table 100 and the patient information acquired from the host computer 500, and generates warning information if the identification information and the patient information do not match each other. In this way, it is possible to prevent a mix-up of patients 3.

Also, in a first embodiment, as explained above, the identification information may be a barcode attached to the patient identification band 3a or may be stored in an RFID tag attached to the patient identification band 3a. Moreover, the identification-information acquisition device 70 may be the barcode reader 70a or the RFID reader 70b. Thus, it is possible to prevent a mix-up of patients 3 with the barcode or the RFID tag.

Also, in a first embodiment, as explained above, the one end of the robotic arm 20 is supported on the base 21, fixed to the floor, while the opposite end supports the table 10. Moreover, the one end of the robotic arm 20 is supported on the base 21 to be rotatable about an axis extending in the vertical direction. Also, the opposite end of the robotic arm 20 supports the table 10 at a position near its one end in the longitudinal direction of the table 10. Furthermore, the robotic arm 20 moves the table 10 with seven degrees of freedom. In this way, the table 10 can be easily moved to desired positions by the robotic arm 20.

Also, in a first embodiment, as explained above, the table 10 includes the radiolucent part 11 and the support part 12, disposed on the one end side of the table 10 in the longitudinal direction of the table 10 and supporting the radiolucent part 11. Moreover, the opposite end of the robotic arm 20 supports the support part 12. In this way, it is possible to minimize the portion of the robotic arm 20 disposed around the radiolucent part 11. Hence, it is possible to leave a sufficient space to place the radiographic imaging apparatus 4 around the radiolucent part 11.

### Second Embodiment

Next, a second embodiment is explained with reference to FIG. 1 and FIG. 7 to FIG. 10. A second embodiment may be explained using an example in which a scheduled surgery end time is updated in addition to what has been explained above in a first embodiment. Components identical to those described above in a first embodiment may be illustrated in the drawings with the same reference sign, and therefore a detailed explanation thereof is omitted for brevity.

### Configuration of Surgery-Progress Management System

A surgery-progress management system 801 according to a second embodiment differs from the surgery-progress management system 1 in a first embodiment in that the surgery-progress management system 801 includes a surgery-progress management apparatus 900, as illustrated in at least FIG. 1. Moreover, the surgery-progress management apparatus 900 differs from the surgery-progress management apparatus 200 in a first embodiment in that the surgery-progress management apparatus 900 includes an information processing unit 992.

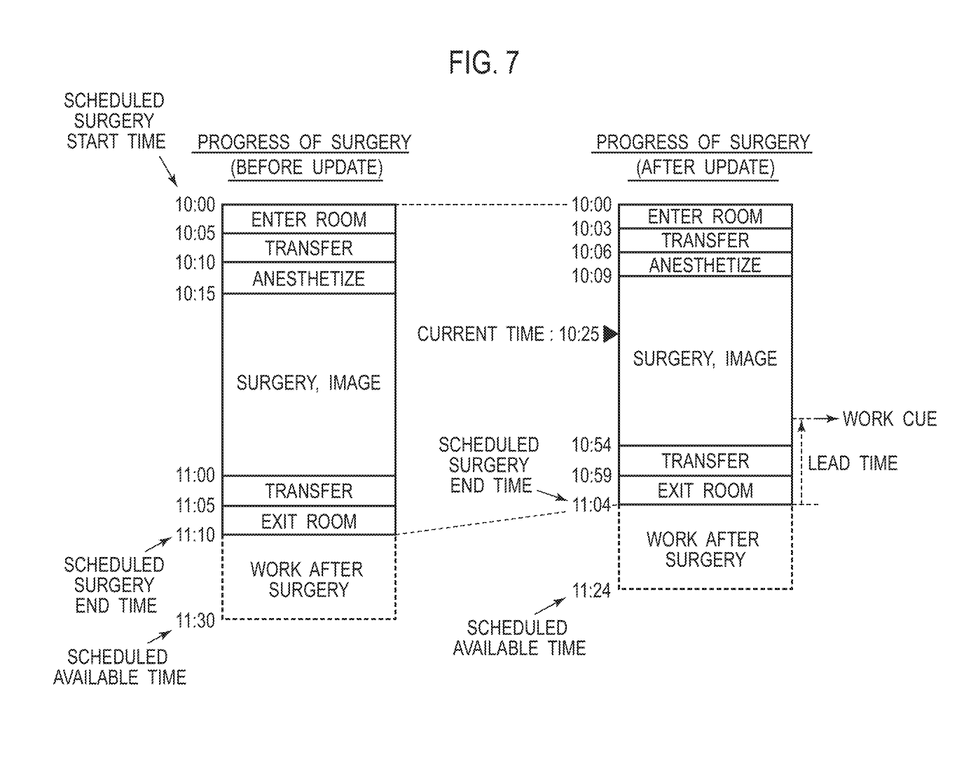

The information processing unit 992 of the surgery-progress management apparatus 900 generates surgery progress information based on at least position information on a table 10, as in a first embodiment. Also, in a second embodiment, the information processing unit 992 sequentially updates a scheduled surgery end time based on the generated surgery progress information, as illustrated in at least FIG. 7.

Specifically, the information processing unit 992 acquires the amount of time actually taken by each process in the surgery based on the generated surgery progress information. The information processing unit 992 also acquires a scheduled surgery start time and the amount of time estimated to be taken (amount of time initially estimated to be taken) by each process in the surgery from a host computer 500 through a communication unit 91, and acquires a scheduled surgery end time based on the scheduled surgery start time and the amount of time to be taken by each process in the surgery. The information processing unit 992 also compares the acquired amount of time actually taken by each process in the surgery and the acquired amount of time initially estimated to be taken by the process in the surgery, and sequentially updates the scheduled surgery end time based on the result of the comparison of the amount of time actually taken and the amount of time initially estimated to be taken. The processes in the surgery may include a process of entering the room, a transfer process before the surgery, an anesthetizing process, a surgery process, an imaging process, a transfer process after the surgery, and a process of existing the room. The processes in the surgery may differ based on the surgical method.

The information processing unit 992 may further performs control for displaying the updated scheduled surgery end time on a display 93. In this way, a medical person (e.g. operator) or other person, may determine the scheduled surgery end time reflecting the actual progress in the room in which the surgery-progress management apparatus 900 is installed. The information processing unit 992 may further transmit the updated scheduled surgery end time to a display 300 and an external device 700 through the communication unit 91. The display 300 and the external device 700 display the received scheduled surgery end time. In this way, a medical person (e.g. operator) or other person, such as a worker (e.g. cleaner), may determine the scheduled surgery end time reflecting the actual progress in the room in which the display 300 is installed, and a worker having the external device 700 can determine the scheduled surgery end time reflecting the actual progress with the external device 700. Meanwhile, the surgery-progress management apparatus 900 provides the updated scheduled surgery end time through the communication unit 91 to the external device 700 in response to a request therefrom.

As illustrated in at least FIG. 7, when the scheduled surgery end time is displayed, the current time, the start time and the end time of each process in the surgery that has been performed, and a scheduled start time and a scheduled end time of each process in the surgery to be performed, and the scheduled surgery end time may be displayed on a time-series graph or in other graphical form.

In a second embodiment, the information processing unit 992 may further transmit a work cue containing the updated scheduled surgery end time to a predetermined informing destination (e.g. an external device 700 of a worker, such as a cleaner) through the communication unit 91. For example, the work cue may be a cue for performing work such as cleaning or maintenance of an operating room 2. Specifically, the information processing unit 992 may transmit a work cue containing the updated scheduled surgery end time to a predetermined informing destination when the amount of time remaining from the current time until the scheduled surgery end time is equal to or shorter than a predetermined lead time.

In a second embodiment, the information processing unit 992 may further transmit a scheduled time from which the operating room 2 will be available to a predetermined informing destination (e.g. an external device 700 of a medical person who will perform the next surgery) through the communication unit 91. The scheduled time from which the operating room 2 will be available is a time calculated by adding the amount of time to be taken by work after the surgery, such as cleaning and maintenance, to the scheduled surgery end time.

Meanwhile, as illustrated in at least FIG. 7, in a case of displaying the above scheduled available time, the amount of time to be taken by the work after the surgery, such as cleaning and maintenance, and the estimated available time may be further displayed on the time-series graph mentioned above, for example.

### Surgery-Information Generation Process

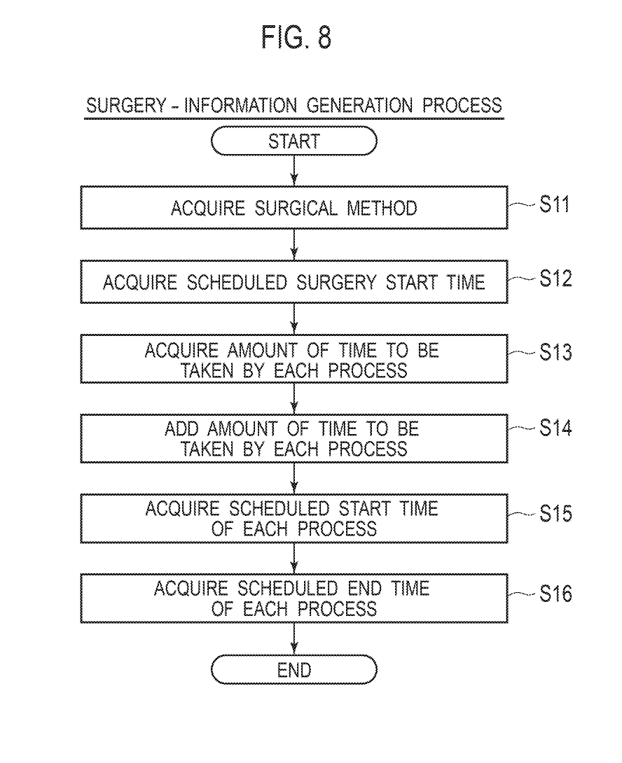

Next, a surgery-information generation process by the surgery-progress management apparatus 900 in a second embodiment is explained with reference to a flowchart in FIG. 8. The information processing unit 992 may perform each process step in the flowchart.

As illustrated in FIG. 8, in step S11, the surgical method is acquired from the host computer 500 through the communication unit 91.

In step S12, the scheduled surgery start time may be acquired from the host computer 500 through the communication unit 91.

In step S13, each process in the surgery may be acquired based on the surgical method acquired in step 51 from the host computer 500 through the communication unit 91. In addition, the amount of time to be taken (amount of time initially estimated to be taken) by each acquired process in the surgery may be acquired from the host computer 500 through the communication unit 91.

In step S14, the amount of time to be taken by each process in the surgery is added.

In step S15, a scheduled start time of each process in the surgery is acquired.

In step S16, a scheduled end time of each process in the surgery and a scheduled surgery end time are acquired. The surgery-information generation process is terminated.

### Scheduled-End-Time Update Process

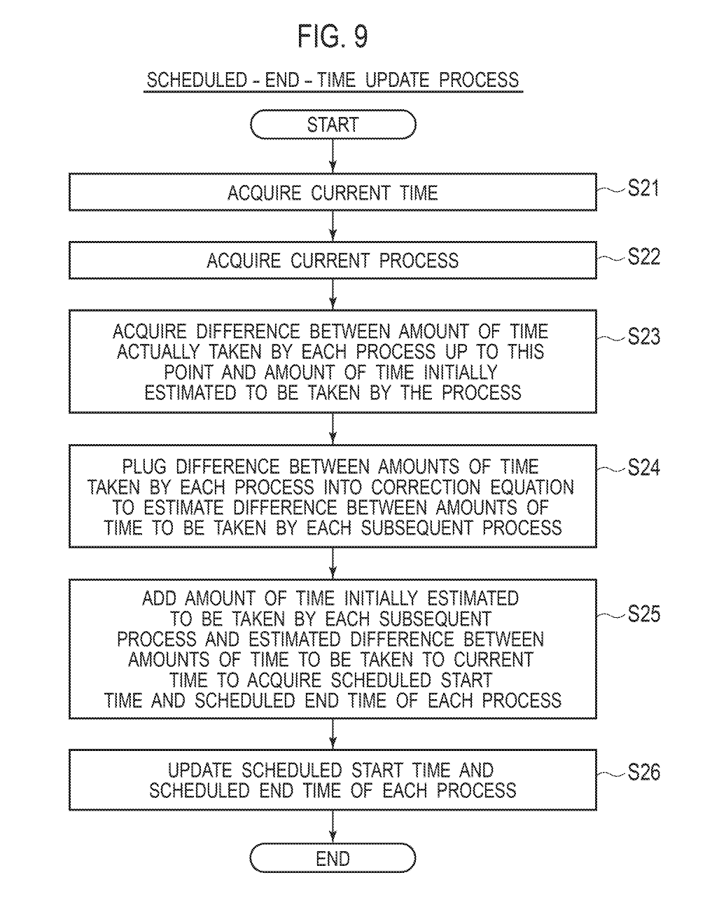

Next, a scheduled-end-time update process by the surgery-progress management apparatus 900 in a second embodiment is explained with reference to a flowchart in FIG. 9. The information processing unit 992 may perform each process step in the flowchart.

As illustrated in FIG. 9, in step S21, the current time is acquired.

In step S22, the current process in the surgery is acquired based on the position information on the table 10 and the like.

In step S23, the difference between the amount of time actually taken by each process in the surgery up to the present or current point in time and the amount of time initially estimated to be taken by the process is acquired.

In step S24, the difference between the above amounts of time taken by each process in the surgery is plugged into a correction equation to estimate the difference between the amounts of time to be taken by each subsequent process in the surgery.

In step S25, the amount of time initially estimated to be taken by each subsequent process in the surgery and the estimated difference between the amounts of time to be taken are added to the current time to thereby acquire a scheduled start time and a scheduled end time of each subsequent process in the surgery and a scheduled surgery end time.

In step S26, the scheduled start time and the scheduled end time of each process in the surgery and the scheduled surgery end time are updated. The scheduled-end-time update process is then terminated.

### Work-Cue Informing Process

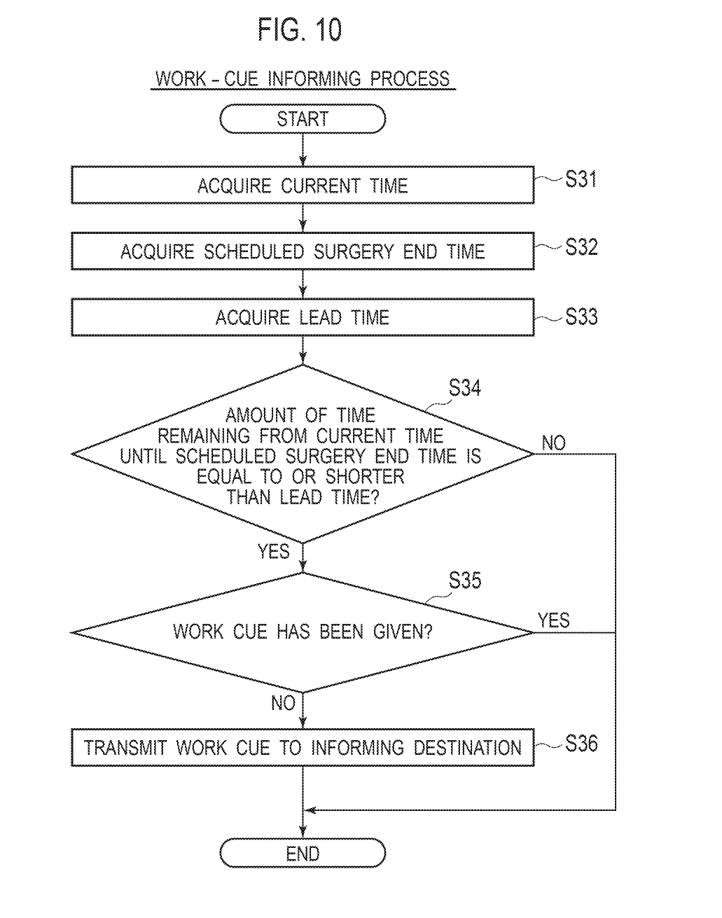

Next, a work-cue informing process by the surgery-progress management apparatus 900 in a second embodiment is explained with reference to a flowchart in FIG. 10. The information processing unit 992 may perform each process step in the flowchart.

As illustrated in at least FIG. 10, in step S31, the current time is acquired.

In step S32, the scheduled surgery end time is acquired.

In step S33, the predetermined lead time is acquired.

In step S34, it is determined whether or not the amount of time remaining from the current time until the scheduled surgery end time is equal to or shorter than the lead time. If it is determined that the amount of time remaining is longer than the lead time (S34=“NO”), it is determined that no work cue needs to be given yet, and the work-cue informing process is therefore terminated. On the other hand, if it is determined the amount of time remaining is equal to or shorter than the lead time (S34=“YES”), the process proceeds to step S35.

In step S35, it is determined whether or not a work cue has been given. If it is determined that a work cue has been given (S35=“YES”), the work-cue informing process is terminated. On the other hand, if it is determined that no work cue has been given (S35=“NO”), the process proceeds to step S36.

In step S36, a work cue containing the scheduled surgery end time is transmitted to an informing destination. As a result, the work cue containing the scheduled surgery end time is displayed at the informing destination. The work-cue informing process is then terminated.

Note that the other features of the configuration in a second embodiment are similar to those in a first embodiment.

### Advantageous Effects of Second Embodiment

Second embodiment can offer the following advantageous effects.

In a second embodiment, as explained above, the information processing unit 992 sequentially updates the scheduled surgery end time based on generated surgery progress information. In this way, a worker who, for example, cleans the operating room 2 can accurately determine an appropriate time to stand by for the cleaning based on the sequentially updated scheduled surgery end time.

Also, in a second embodiment, as explained above, the information processing unit 992 transmits a work cue containing the updated scheduled surgery end time to a predetermined informing destination through the communication unit 91. In this way, a worker who, for example, cleans the operating room 2 can determine a more appropriate time to stand by for the cleaning without excessive effort.

Also, in a second embodiment, as explained above, the information processing unit 992 transmits a scheduled time from which the operating room 2 will be available to a predetermined informing destination through the communication unit 91. In this way, a worker who, for example, will perform the next surgery can determine an appropriate time to stand by for the next surgery without excessive effort.

Also, in a second embodiment, as explained above, the information processing unit 992 acquires the scheduled surgery start time from the host computer 500. In this way, the scheduled surgery start time does not need to be manually input into the surgery-progress management apparatus 900, which can reduce the work burden on the user.

Note that other advantageous effects of second embodiment are similar to those of a first embodiment.

### Modifications

The embodiments disclosed herein should be considered exemplary in all aspects, non-exhaustive and not limiting. The scope of the present invention is indicated by the claims rather than the explanation of the above embodiments and also embraces all changes that come within the meaning and range of equivalents of the claims.

For example, although the example with the configuration in which a radiographic imaging apparatus is provided in a hybrid operating room has been presented in a first embodiment and a second embodiment, additional or alternative embodiments may not be limited to such examples. For example, a magnetic resonance imaging apparatus that captures a magnetic resonance image of a patient may be provided in a hybrid operating room in accordance with one or more embodiments. Further, both a radiographic imaging apparatus and a magnetic resonance imaging apparatus may be provided in a hybrid operating room in accordance with one or more embodiments.

Moreover, although in the above described examples, a configuration in which the robotic operating table is provided in a hybrid operating room has been presented in a first embodiment and a second embodiment, additional or alternative embodiments are not limited to these examples. For example, a robotic operating table may be provided in an operating room other than a hybrid operating room.

Further, although the above described examples in which the information processing unit of the surgery-progress management apparatus generates surgery progress information based on the load information, the result of the detection by the load sensor, and the result of the detection by the door sensor in addition to the position information on the table have been presented in a first embodiment and a second embodiment, additional or alternative embodiments are not limited to these examples. For example, the load information, the result of the detection by the load sensor, and the result of the detection by the door sensor may not be used as long as suitable surgery progress information can be generated.

Further, although the above described examples in which the position information on the table includes the home-position information, the transfer-position information, the anesthetization-position information, the imaging-position information, and the surgery-position information have been presented in a first embodiment and a second embodiment, additional or alternative embodiments are not limited to these examples. For example, the position information on the table may not include all of the home-position information, the transfer-position information, the anesthetization-position information, the imaging-position information, and the surgery-position information or may contain position information on the table other than the above described information.

Further, although the above described example in which the home position, the transfer position, the anesthetization position, the imaging position, and the surgery position are registered as preset positions in the robotic operating table has been presented in a first embodiment and a second embodiment, additional or alternative embodiments are not limited to these examples. For example, a cleaning position for performing cleaning may be registered as a preset position. In this case, the position information on the table and the posture information on the robotic arm 20 in a state where, for example, the robotic arm 20 is placed in such a posture as to raise the table to the highest level at the home position, may be registered as the cleaning position. Also, present positions may not be registered in the robotic operating table.

Further, although the above described example in which the robotic operating table, the surgery-progress management apparatus, the display, the host computer, the door sensor, and the external device are communicatively connected to each other through an LAN has been presented in a first embodiment and a second embodiment, additional or alternative embodiments are not limited to such examples. For example, the robotic operating table, the surgery-progress management apparatus, the display, the host computer, the door sensor, and the external device may be communicatively connected to each other without an LAN.

Further, although the above described example in which the surgery-progress management system includes a display has been presented in a first and second embodiments, additional or alternative embodiments are not limited to such examples. For example, the surgery-progress management system may not include a display.

Further, although the above described example in which the information processing unit of the surgery-progress management apparatus acquires the scheduled surgery start time from the host computer has been presented in a first embodiment and a second embodiment, additional or alternative embodiments are not limited to such examples. For example, the information processing unit may acquire the scheduled surgery end time from the host computer. Also, the information processing unit may acquire both the scheduled surgery start time and the scheduled surgery end time from the host computer.

Further, although in the above described example, a configuration in which the horizontal articulated assembly includes three horizontal joints has been presented in a first embodiment and a second embodiment, additional or alternative embodiments are not limited to such examples. For example, the horizontal articulated assembly may include two horizontal joints or include four or more horizontal joints.

Further, although the above described example Also, although the example with the configuration in which the vertical articulated assembly includes three vertical joints has been presented in a first embodiment and a second embodiment, additional or alternative embodiments are not limited to such examples. For example, the vertical articulated assembly may include two vertical joints or include four or more vertical joints.

Further, although the above described example Also, although the example with the configuration in which the robotic arm 20 has seven degrees of freedom has been presented in a first embodiment and a second embodiment, additional or alternative embodiments are not limited to such examples. For example, the robotic arm 20 may have six or fewer degrees of freedom or have eight or more degrees of freedom. However, it is preferable for the robotic arm 20 to have six or more degrees of freedom.

Further, although the above described example Also, although the example with the configuration in which the base is buried in and fixed to the floor has been presented in a first embodiment and a second embodiment, additional or alternative embodiments are not limited to such examples. For example, the base may be fixed to the surface of the floor.

Patent Literature 1 describes related art in which an instrument may be used throughout surgery while the state of the instrument changes during the progress of the surgery. However, it is difficult to accurately determine the progress of surgery in real time by only detecting the on and off states of the power sources for the instruments described in Patent Literature 1. As a result, it is difficult for a worker, such as a cleaner, who cleans an operating room, to determine an appropriate time to stand by for the cleaning.

The embodiments described above is directed to a surgery-progress management system and a surgery-progress management apparatus which make it possible to accurately determine the progress of surgery in real time.

The above-described aspects may be combined with each other as practicable within the contemplated scope of embodiments. The above described embodiments are to be considered in all respects as illustrative, and not restrictive. The illustrated and described embodiments may be extended to encompass other embodiments in addition to those specifically described above without departing from the intended scope of the invention. The scope of the invention is to be determined by the appended claims when read in light of the specification including equivalents, rather than solely by the foregoing description. Thus, all configurations including configurations that fall within equivalent arrangements of the claims are intended to be embraced in the invention.

What is claimed is:

1. A surgery-progress management system comprising:
a robotic operating table comprising a table top on which to place a patient and a robotic arm, which comprises a plurality of joints and supports the table top; and
a surgery-progress management apparatus that manages progress of surgery,
wherein
the surgery-progress management apparatus comprises:
a communication unit that communicates information with the robotic operating table; and
an information processing unit that acquires information of the robotic operating table from the robotic operating table through the communication unit and generates surgery progress information based on the acquired information of the robotic operating table.

2. The surgery-progress management system according to claim 1, wherein
the information of the robotic operating table comprises position information of the table top, and
the information processing unit generates surgery progress information based on the acquired positon information of the table top.

3. The surgery-progress management system according to claim 2, wherein the position information of the table top comprises patient-receiving position information indicating that the table top has been placed at a patient-receiving position for receiving the patient from a stretcher to the table top.

4. The surgery-progress management system according to claim 2, wherein the position information of the table top comprises at least one of
anesthetization-position information indicating that the table top has been placed at an anesthetization position for anesthetizing the patient,
imaging-position information indicating that the table top has been placed at an imaging position for capturing an image with a radiographic imaging apparatus or a magnetic resonance imaging apparatus, and
surgery-position information indicating that the table top has been placed at a surgery position for performing surgery.

5. The surgery-progress management system according to claim 2, wherein the position information includes transfer-position information indicating that the table top has been placed at a transfer position for transferring the patient from the table top to a stretcher, and the information processing unit generates information indicating that the surgery is ended soon, as the surgery progress information based on the transfer-position information.

6. The surgery-progress management system according to claim 1, wherein the robotic operating table is configured to register a position of the table top as a preset position.

7. The surgery-progress management system according to claim 4, wherein the robotic operating table is configured to register at least one of the anesthetization position, the imaging position, and the surgery position as a preset position.

8. The surgery-progress management system according to claim 6, wherein the robotic operating table registers a predetermined position as a preset position in response to a register command issued with the table top placed at the predetermined position.

9. The surgery-progress management system according to claim 2, wherein
each of the joints of the robotic arm comprises a motor and an encoder that measures an amount of rotation of the motor, and
the robotic operating table generates the position information of the table top based on a result of the measurement by the encoder.

10. The surgery-progress management system according to claim 9, wherein
the robotic operating table acquires a current value of the motor of each of the joints of the robotic arm and generates load information on a load received by the robotic arm from the table top based on a result of the acquisition of the current value of the motor, and
the information processing unit acquires the load information from the robotic operating table through the communication unit and generates the surgery progress information based on the acquired load information in addition to the position information of the table top.

11. The surgery-progress management system according to claim 2, wherein
the robotic operating table comprises a load sensor for detecting a load that the table top receives from the patient, and
the information processing unit acquires a result of the detection by the load sensor from the robotic operating table through the communication unit and generates the surgery progress information based on the acquired result of the detection by the load sensor in addition to the position information of the table top.

12. The surgery-progress management system according to claim 1, wherein
the information processing unit acquires a result of detection by a door sensor that detects opening and closing of a door of an operating room, through the communication unit, and generates the surgery progress information based on the result of the detection by the door sensor in addition to the information of the robotic operating table.

13. The surgery-progress management system according to claim 1, wherein the surgery-progress management apparatus provides the surgery progress information through the communication unit to an external device in response to a request therefrom.

14. The surgery-progress management system according to claim 1, wherein the surgery-progress management apparatus is communicatively connected to a host computer through the communication unit and is configured to acquire patient information from the host computer.

15. The surgery-progress management system according to claim 14, wherein
the robotic operating table comprises an identification-information acquisition device that acquires identification information attached to the patient, and
the information processing unit compares the identification information acquired from the robotic operating table and the patient information acquired from the host computer, and generates warning information if the identification information and the patient information do not match each other.

16. The surgery-progress management system according to claim 15, wherein
the identification information is included in a barcode attached to a patient identification band or is stored in an RFID tag attached to the patient identification band, and
the identification-information acquisition device is a barcode reader or an RFID reader.

17. The surgery-progress management system according to claim 14, wherein the information processing unit acquires a scheduled surgery start time or a scheduled surgery end time from the host computer.

18. The surgery-progress management system according to claim 1, wherein
one end of the robotic arm is supported on a base fixed to a floor while an opposite end of the robotic arm supports the table top,
the one end of the robotic arm is supported on the base to be rotatable about an axis extending in a vertical direction,
the opposite end of the robotic arm supports the table top at a position near one end in a longitudinal direction of the table top, and
the robotic arm moves the table top with at least six degrees of freedom.

19. The surgery-progress management system according to claim 1, wherein
the table top comprises a radiolucent part and a support part disposed on a one end side of the table top in a longitudinal direction of the table top and supporting the radiolucent part, and
the opposite end of the robotic arm supports the support part.

20. A surgery-progress management apparatus comprising:
a communication unit that communicates information with a robotic operating table including a table top on which to place a patient and a robotic arm which comprises a plurality of joints and supports the table top; and
an information processing unit that acquires information of the robotic operating table from the robotic operating table through the communication unit and generates surgery progress information based on the acquired information of the robotic operating table.
